(19) United States
(12) Patent Application Publication
(10) Pub. No.: US 2016/0245832 A1
(43) Pub. Date: Aug. 25, 2016

(54) Biological chip systems

(71) Applicant: Kwong Hou WONG, Hong Kong (HK)

(72) Inventor: Kwong Hou WONG, Hong Kong (HK)

(21) Appl. No.: 14/631,780

(22) Filed: Feb. 25, 2015

(51) Int. Cl. G01N 35/00; G01N 35/10; C12Q 1/68

(52) U.S. Cl. CPC G01N 35/00029; C12Q 1/6837; G01N 35/1081; G01N 35/1065; G01N 2035/00158; G01N 2035/00376; G01N 2035/00346

(57) ABSTRACT

A biological chip system is provided in which execution, cleaning, scanning, and/or drying are all integrated and automated. According to one embodiment, the system employs an execution system, a first mobile structure movable, a second mobile structure, a third mobile structure, at least two fluid paths, a fluid control system, a cleaning device, a biochip scanning system, and/or a computer control system. The computer control system processes co-ordination among the biochip scanning system, the cleaning device, the fluid control system, and/or the execution system.

### FIELD OF THE INVENTION

The present invention relates to a biological system, particularly to an integrated system based on a biological chip hardware system that processes co-ordination among a biochip scanning system, a cleaning device, a fluid control system, and an execution system.

### BACKGROUND OF THE INVENTION

Current operations of biochip systems go through multiple independent steps to process molecules to determine whether certain coded instructions for the cells are to be found. In the process, the steps require a plurality of procedures, including loading, rinsing, mixing, heating, cooling, cleaning, and/or scanning imaging. The entire process typically involves a variety of equipment and instruments, so that the detection efficiency can be very undesirable, and can be subject to human inefficiency and even human errors. It is certainly more desirable to complete the testing process in a single integration, as well as the preparation or sampling process in an automated manner in order to maximize the productivity while reducing costs.

For biochip systems to perform properly, there is a need to use a range of operating and testing equipment, production apparatuses, and connection control equipment. Further, it is desirable to test all of the operations, electronic control device production apparatus and computer control systems, in order to improve the mechanical and electrical aspects in order to properly and efficiently achieve a fully automated detection process.

There have been some prior art references directed towards biochip systems for building biochip platforms. Yet, most of these systems cannot be carried out entirely on a single device. Further, the processes disadvantageously require manual assistance to transfer the samples across several difference pieces of processing equipment. From that point of view, automation is undesirable. Based on the description above, the present biological chip system has disadvantages such as long hybridization duration, low efficiency, complicated operational procedure, and low integration and automation levels.

### SUMMARY OF THE INVENTION

Based upon the aforementioned problems, the objective of the present invention is to provide a biological chip system in which execution, cleaning, scanning, and/or drying are all integrated and automated. According to one embodiment, a biological system uses an execution system, a first mobile structure, a second mobile structure, a third mobile structure, at least two fluid paths, a fluid control system, a cleaning device, a biochip scanning system, and/or a computer control system that processes co-ordination among the biochip scanning system, the cleaning device, the fluid control system, and/or the execution system. The system defines a horizontal co-ordinate, a vertical co-ordinate, and/or a co-ordinate perpendicular to the horizontal co-ordinate and the vertical co-ordinate. The execution system includes a biochip microarray, which employs an underlying substance with a probe and a plate with at least two through hollow portions. A hybridization area is formed between the underlying substance and the plate.

In one embodiment, the chip system may include a first mobile structure movable along the horizontal axis, a second mobile structure movable along the vertical axis, and/or a third mobile structure movable along the axis perpendicular to the horizontal and vertical axis. Further, at least two fluid paths are included in the embodiment, which are connected to the hybridization area through the two hollow places, respectively.

In another aspect of the embodiment, a fluid control system is provided. The fluid control system is connected to the hybridization area through the two hollow places, respectively.

In yet another aspect of the embodiment, a cleaning device is attached to the first mobile structure, and a biochip scanning system is attached to the third mobile structure.

An object of the present invention is to integrate, automate and streamline the processes carried out by the steps of preparing, sampling, testing, and/or validating molecules that contain coded instructions for the cells. As such, a computer control system is introduced as an embodiment to process co-ordination among the biochip scanning system, the cleaning device, the fluid control system, and/or the execution system.

In one embodiment, the fluid control system comprises at least one gas provider for providing gas pressure, and one fluid regulator for controlling the entrance of the fluid. The fluid provider is connected to the fluid path through the fluid regulator. The gas provider and the fluid regulator are placed on the second mobile structure and the third mobile structure.

Advantageously, in another embodiment, the probe of the biochip microarray includes a molecule that contains coded instructions. A number of different material may be used in the present invention. In one embodiment, the plate of the biochip microarray may be composed of silicon polymers, whereas the material for the underlying substance of the biochip microarray may be glass, silicon, high molecular polymer and/or any combination thereof.

Another embodied system feature is directed to a temperature control system. The temperature control system employs a hot plate, a heating body, a cooling body, a temperature sensor, and/or a temperature controller. The material for the hot plate may be metal or alloy, and/or non-metallic materials with high coefficients of heat conductivity, such as, for example, silicon or glass.

### BRIEF DESCRIPTION OF THE DRAWINGS

FIG. 1 is a schematic illustration of the computer control system, the biochip scanning system, the cleaning device, the fluid control system, and the execution system, according to an embodiment of the present invention.

FIG. 2 is a top view of a biochip microarray according to an embodiment of the present invention.

FIG. 3 is a block diagram showing a system chassis according to an embodiment of the present invention.

FIG. 4 is a perspective view showing the system chassis of according to an embodiment of the present invention.

FIG. 5 is a high-level block diagram of a microprocessor device that may be used to carry out the disclosed technology.

### DETAILED DESCRIPTION

References will now be made in detail to the present exemplary embodiments, examples of which are illustrated in the accompanying drawings. Whenever possible, the same reference numbers will be used throughout the drawings to refer to the same or like parts.

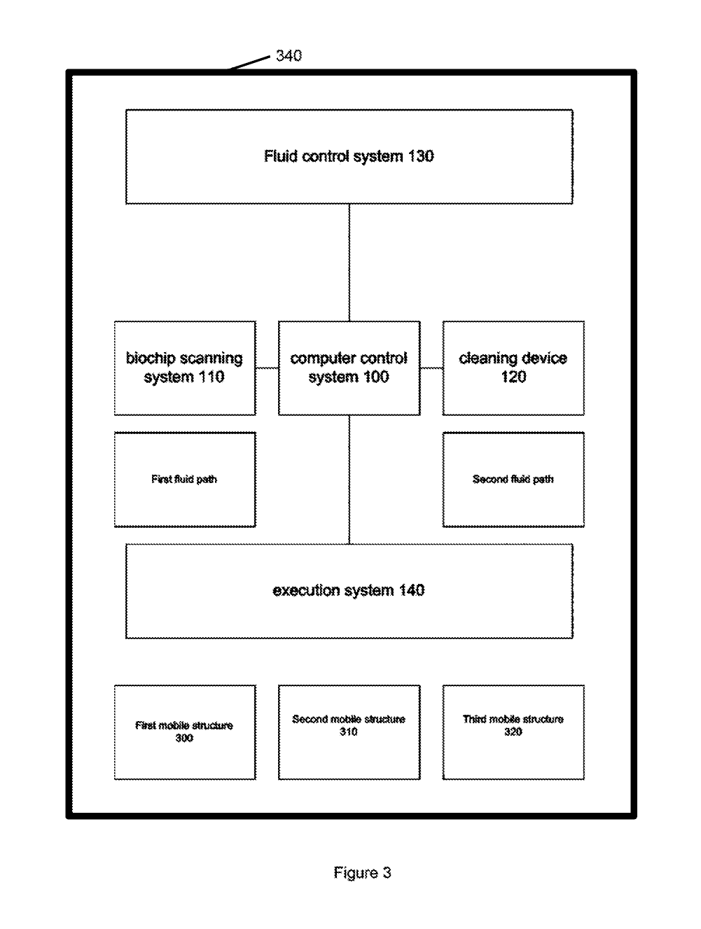

FIG. 3 shows an overall arrangement of a computer control system, a biochip scanning system 110, a cleaning device 120, a fluid control system 130, and the execution system 140 according to an embodiment of the present invention. In the example, a computer control system 100 processes information flow between the aforementioned sub-systems.

Biochip scanning systems 110 have been integral for biomedical applications and many such systems are known in the art. Optical detection systems have been a very important tool in biochip analysis. Microscopes are often inadequate for high resolution and big view-area detection of microarray chips 210, however, some new optical instruments may be required to suit differing needs. As such, a novel digital imaging scanning system with dark-field imaging has been developed for some biomedical applications for microarray chips 210. The system is characterized by analyzing genes and proteins of clinical samples with high specific samples. The novel optical system has a high numerical aperture, a long working distance, an excellent contrast and signal-to-noise ratio, a high resolving power, and an efficiency of fluorescence more than two-fold better than that of other commercial biochip scanners. An edge overlap algorithm is proposed for the image restructure of free area detection and correcting scanning position errors to a precision of small pixels. A novel algorithm is explored for recognizing the target from the scanning images conveniently, removing noise, and/or producing the signal matrix of biochip analysis. The digital imaging scanning system is equally as good for the detection of enclosed biochips as it is for the detection of biological samples on a slide surface covered with a glass cover slip or in a culture solution.

Cleaning device 120 refers to a device that removes contaminants on the surface of a biochip. These contaminants are generally cleaned by either a wet cleaning method or a dry cleaning method. In the wet cleaning method, chemicals are used for dipping and washing the chip. Most users adopt the wet cleaning method. The wet cleaning method requires a multitude of liquid chemicals, including organic solvents and inorganic solvents or even strong acidic or alkaline solutions, and thus the wet cleaning method may create secondary pollution easily or cause poison leaking or exposure during the cleaning process. Furthermore, there are the matters of recycling chemical waste and requiring a very clean environment for rinsing the biochips. The wet cleaning method is unable to achieve a high standard and causes inconvenience to users, and thus the dry cleaning method needs to be properly implemented.

A fluid control system 130 is connected to the hybridization area through two hollow places, respectively. The fluid control system 130 employs at least one gas provider for providing gas pressure. In one example, a pump is used to provide gas pressure. The fluid control system 130 may also include a fluid regulator for controlling the entrance of the fluid. A fluid provider is connected to the fluid path through the fluid regulator. Further, the gas provider and the fluid regulator are placed on the second mobile structure and the third mobile structure, respectively. In an implementation, one example of deploying the fluid regulator is by way of a valve.

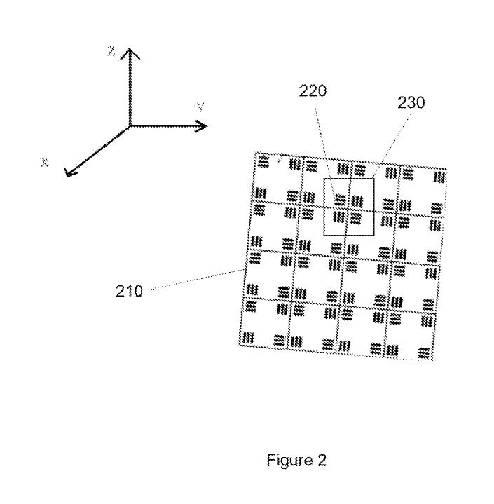

Referring now to FIG. 2, a top view of a biochip microarray is depicted according to an embodiment of the present invention. The microarray depicted in FIG. 2 is a detailed view of the execution system 140. It includes a biochip microarray 210, which utilizes an underlying substance with a probe 220 and a plate 230 with at least two through hollow places. A hybridization area is formed between the underlying substance and the plate. In one example, the underlying substance can refer to a substrate while the plate can be implemented as a cover plate. Generally, a “probe”, when referred to this example, may be a device that is commonly called a “probe array”, which is collection of DNA spots attached to a solid surface in which the DNA molecules cannot be seen macroscopically.

To improve efficiency, it is desirable to have an integrated system that can also automate the processes among all the subsystems that are used in a biochip system. This is formed with a system chassis 340 that serves as a backbone linking all the other major components.

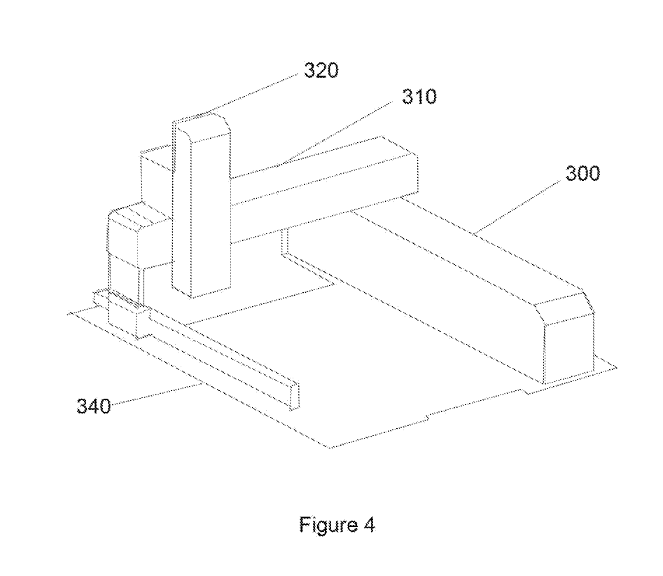

FIG. 4 is a perspective view showing the system chassis of according to an embodiment of the present invention. FIG. 4 illustrates a system chassis 340 including, among others, an execution system 140 defining a horizontal co-ordinate, a vertical co-ordinate; and a co-ordinate perpendicular to the horizontal co-ordinate and the vertical co-ordinate. With the co-ordinates fixed in place, system chassis 340 further provides movable structures that may slide along the above three co-ordinates.

In one example, a first mobile structure 300 is movable along the horizontal co-ordinate as shown; a second mobile structure 310 is movable along the horizontal co-ordinate as shown; and a third mobile structure 320 movable along the another co-ordinate as shown. In the example, a cleaning device 120 is attached to first mobile structure 300 whereas biochip scanning system 110 is attached to the third mobile structure 320. Further, the gas provider and the fluid regulator are placed on the second mobile structure 310 and the third mobile structure 320, respectively.

Additionally, temperature control is integral to the biochip system. As such, the biological chip system according to the present invention further employs a temperature control device. The temperature control device may have one or more of the following components: a hot plate, a heating body, a cooling body, a temperature sensor and/or a temperature controller. The material which composes the hot plate may be metal or alloy, and/or may also be non-metallic materials with large coefficients of heat conductivity, such as, for example, silicon or glass. The hot plate is used for placing the chip and conducting heat. The temperature control mode therefore may be temperature control of the upper surface and/or the lower surface.

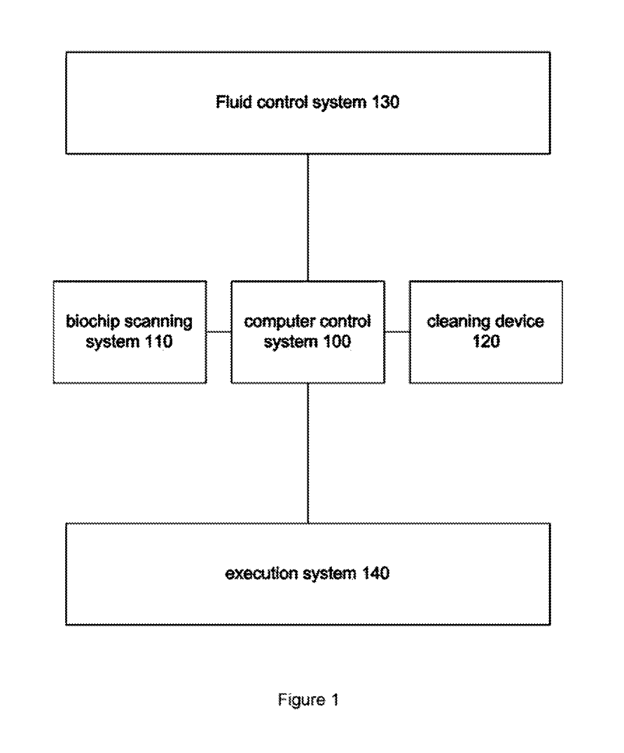

FIG. 1 is a schematic illustration of the computer control system, the biochip scanning system, the cleaning device, the fluid control system, and the execution system, according to an embodiment of the present invention. Computer control system 100 serves as the controlling unit of the biological chip system. In an example of the present invention, a computer control system 100 uses a control circuit. The control circuit in this example uses a single computer system, but in practice, any computing systems with adequate computer power may be included for the purposes of this specification and/or any real world application. The control unit automatically controls the opening and closing of the valve, operation of the pump, and/or the temperature of the heater. Instruction is given by the control circuit to the temperature control device, so that the temperature of the chip and the sample solution can be automatically controlled. Instruction is also given by the control circuit to the control device, in order to automatically control the fluid during the chip hybridization, cleaning and drying processes. Powered by the pump valve device, the cleaning liquid and air can be injected from the fluid storing device to the hybridization chamber through the fluid channel, and then discharged to the storage device through the fluid channel.

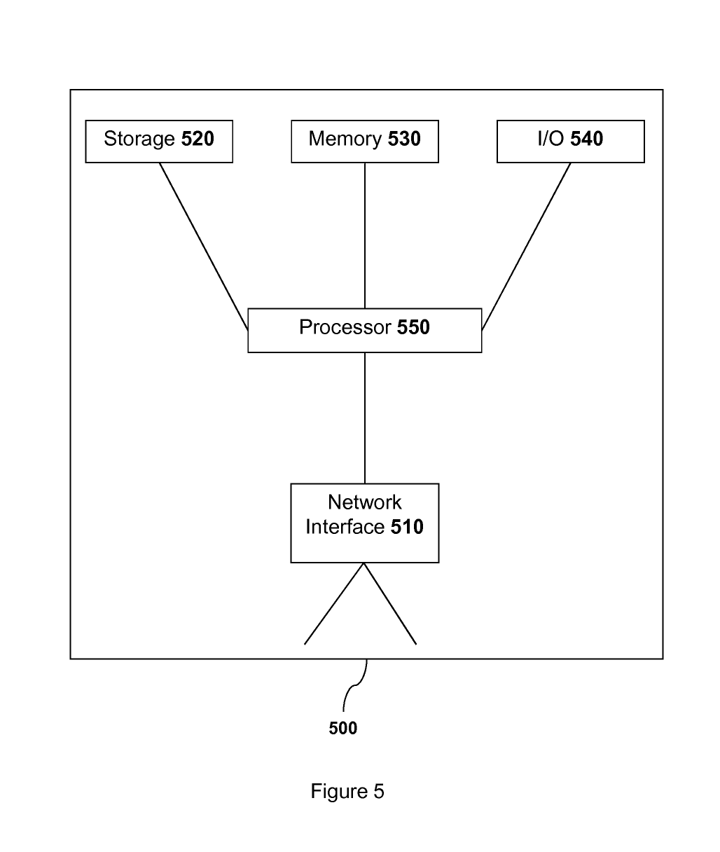

FIG. 5 is a high-level block diagram of a microprocessor device that may be used to carry out the disclosed technology. The device 500 may be employed to control electronic or automated components of the disclosed technology. The device 500 comprises a processor 550 that controls the overall operation of a computer by executing the reader's program instructions which define such operation. The reader's program instructions may be stored in a storage device 520 (e.g., magnetic disk, database) and loaded into memory 530 when execution of the console's program instructions is desired. Thus, the device 500 will be defined by the program instructions stored in memory 530 and/or storage 520, and the console will be controlled by processor 550 executing the console's program instructions.

The device 500 may also include one or a plurality of input network interfaces for communicating with other devices via a network (e.g., the internet). The device 500 further includes an electrical input interface for receiving power and data. The device 500 also includes one or more output network interfaces 510 for communicating with other devices. The device 500 may also include input/output 540 representing devices which allow for user interaction with a computer (e.g., display, keyboard, mouse, speakers, buttons, etc.).

One skilled in the art will recognize that an implementation of an actual device will contain other components as well, and that FIG. 5 is a high level representation of some of the components of such a device for illustrative purposes. It should also be understood by one skilled in the art that the method and devices depicted in FIGS. 1 through 4 may be implemented on a device such as is shown in FIG. 5.

While it is obvious that modification or proper change and combination can be made to the present biological chip system according to the present invention by those skilled in the art, however, without departing from the contents, spirit and scope of the invention, any variations that are intended to achieve the techniques disclosed in the present invention should be within the scope of this invention. Specifically, it should be pointed out that all similar substitutions and modifications are obvious to those skilled in the art, and they are deemed to be within the scope and content of the present invention.

It is to be understood that the foregoing detailed description and accompanying drawings relate to a preferred illustrative embodiment of the invention. However, various changes and modifications may be made without departing from the spirit and scope of the invention. Accordingly, the present invention is not limited to the specific arrangements as shown in the drawings and described in detail herein above. The exemplary materials, constructions and illustrations included in the preferred embodiment and this patent application should therefore not be construed to limit the scope of the present invention, which is defined by the appended claims.

While the disclosed invention has been taught with specific reference to the above embodiments, a person having ordinary skill in the art will recognize that changes may be made in form and detail without departing from the spirit and the scope of the invention. The described embodiments are to be considered in all respects only as illustrative and not restrictive. All changes that come within the meaning and range of equivalency of the claims are to be embraced within their scope. Combinations of any of the methods, systems, and devices described hereinabove are also contemplated and within the scope of the invention.

1. A biological chip system, comprising:
a system chassis that further includes:
biochip microarray, the biochip microarray comprising a substrate with a probe and a cover plate having at least two through hollow places, further wherein a hybridization area is formed between the substrate and the cover plate;
a first mobile structure movable along a horizontal co-ordinate;
a second mobile structure movable along a vertical co-ordinate;
a third mobile structure movable along a co-ordinate perpendicular to the horizontal co-ordinate and the vertical co-ordinate;
at least two fluid paths connected to the hybridization area through said two through hollow places, respectively;
a fluid control system connected to the hybridization area through said two through hollow places, respectively, the fluid control system comprising:
at least one gas provider for providing gas pressure;
a fluid regulator for controlling the entrance of the fluid; and
a fluid provider connected to the fluid path through the fluid regulator, wherein the gas provider is attached to the second mobile structure and the fluid regulator is attached to the third mobile structure;
a cleaning device attached to the first mobile structure;
a biochip scanning system attached to the third mobile structure; and
a computer control system for processing co-ordination. among the biochip scanning system, the cleaning device, and the fluid control system.

2. (canceled)

3. The biological chip system of claim 1, wherein the probe of the biochip microarray includes a DNA molecule.

4. The biological chip system of claim 1, wherein the cover plate of the biochip microarray comprises silicon polymers.

5. The biological chip system of claim 1, wherein the substrate of the biochip microarray comprises glass, silicon, or high molecular polymer.

6. The biological chip system of claim 1, further comprising a temperature control system.

7. The biological chip system of claim 6, wherein the temperature control system comprises a hot plate, a heating body, a cooling body, a temperature sensor, and a temperature controller.

8. The biological chip system of claim 7, wherein the material composing the hot plate is metal, alloy, or a non-metallic material with a high coefficient of heat conductivity.

9. The biological chip system of claim 8, wherein the non-metallic material is glass.

10. The biological chip system of claim 8, wherein the non-metallic material is silicon.
